(19) United States
(12) Patent Application Publication
(10) Pub. No.: US 2011/0284231 A1
(43) Pub. Date: Nov. 24, 2011

(54) Electromagnetic Wave Treatment Of Oil Wells

(75) Inventor: Harold L. Becker, Tomball, TX (US)

(73) Assignee: BAKER HUGHES INCORPORATED, Houston, TX (US)

(21) Appl. No.: 13/147,188

(22) PCT Filed: Oct. 2, 2009
(86) PCT No.: PCT/US09/59411

§ 371 Date: Jul. 29, 2011

Related U.S. Application Data
(63) Continuation of application No. 12/365,750, filed on Feb. 4, 2009.
Application No. 12/365,750 is a continuation of application No. PCT/US09/44353, filed on May 18, 2009.
Application No. PCT/US09/44353 is a continuation of application No. 12/365,750, filed on Feb. 4, 2009.
(60) Provisional application No. 61/221,441, filed on Jun. 29, 2009.
(60) Provisional application No. 61/054,157, filed on May 18, 2008.
(60) Provisional application No. 61/054,157, filed on May 18, 2008.

(51) Int. Cl. E21B 43/00; A61L 2/03; C02F 1/48; B01J 19/12; C02F 1/36

(52) U.S. Cl. 166/310; 250/492.1; 210/708; 210/695; 422/22

(57) ABSTRACT

A method including exposing a substance to a first type of electromagnetic waves generated by a first device. The frequency of the first type of electromagnetic waves is in the radio frequency range and the device preferably consumes no more than about 1,000 Watts of power. The exposure takes place for a period of time and at a frequency sufficient to detectably alter at least one physical property of the substance as it existed prior to the exposure. The substance is selected from the group consisting of a hydrate, a water and oil emulsion, clay, scale, cement, a completion fluid, tank sediment and iron sulfide.

### REFERENCE TO RELATED APPLICATIONS

Claim is hereby made to the benefit of the priority of co-pending PCT International Patent Application No. PCT/US2009/44353, filed May 18, 2009, which in turn claims the benefit of the priority of co-pending U.S. application Ser. No. 12/365,750, filed Feb. 4, 2009, which in turn claims the benefit of the priority of U.S. Provisional Application No. 61/054,157, filed May 18, 2008. Claim is also hereby made to the benefit of the priority of co-pending U.S. Provisional Application No. 61/221,441, filed Jun. 29, 2009. The disclosures of each of the foregoing are incorporated herein by reference.

### TECHNICAL FIELD

This invention relates to a method for altering physical properties of hydrocarbonaceous or other material through the application of electromagnetic waves, specifically radio waves or a combination of radio waves and microwaves.

### THE INVENTION

The present invention provides, amongst other things, a system for, and a method of, altering the composition of a hydrocarbonaceous material by exposing the hydrocarbonaceous material to combination of electromagnetic waves for a time and under conditions sufficient to alter the molecular structure or a physical property of at least one component of the hydrocarbonaceous material. As used herein, the term physical property may include London-Van DerWal forces of induction, hydrogen bonding, waxy paraffin solubility in crude oils, decreased viscosity of complex fluids, oil to water ratios in produced crude oil, morphology, etc. The exposure may be accomplished conveniently through the use of a radio frequency (RF) generator and a RF power amplifier, or through the use of such a RF generator and RF power amplifier in combination with a microwave generator and microwave amplifier combination. The invention enables rapid and economical improvement in the production of hydrocarbon (e.g., gas and/or oil) wells while consuming a relatively lower level of power.

In an embodiment of the present invention, provided is a method comprising exposing a substance to a first type of electromagnetic waves generated by a first device. The frequency of the first type of electromagnetic waves is in the radio frequency range and the device consumes no more than about 1,000 Watts of power. The exposure takes place for a period of time and at a frequency sufficient to detectably alter at least one physical property of the substance as it existed prior to the exposure. Substances exposed for treatment in accordance with this method may include, e.g., hydrocarbonaceous (i.e., hydrocarbon-containing) materials, mineral scale deposits, oil-water emulsions, hydrates and the like. In another aspect of the invention, the substance exposed for treatment is selected from the group consisting of a hydrate, a water and oil emulsion, clay, scale, cement, a completion fluid, tank sediment and iron sulfide. Applications of the invention thus also include at least a method of de-emulsifying an emulsion by applying this method to an emulsion so as to cause oil in the emulsion to separate from water in the emulsion, a method of treating and/or inhibiting hydrate formation by applying this method to a hydrate or a treatment zone where hydrate inhibition is desired so as to reduce the amount of hydrate present, and a method of treating and/or inhibiting scale formation by applying this method to scale deposit(s) or a treatment zone where scale inhibition is desired so as to reduce the amount of scale present. The treatment zone or zones in these applications may include, e.g., a well bore, well casing, production tubing, well formations, well head assemblies, associated pumps (including downhole equipment), storage tanks, pipelines, production equipment and the like. These and other applications of the invention are more fully described below.

In another embodiment of the present invention, provided is a process comprising transmitting electromagnetic waves at one or more radio frequencies through at least one first antenna (i) connected to, or disposed within, a wellhead assembly, well casing or well tubing of a hydrocarbon well; (ii) disposed within a pipeline comprising hydrocarbonaceous material; or (iii) disposed within a tank comprising hydrocarbonaceous material. Each of the radio frequencies is in the range of about 1 to about 900 MHz and amplified to no more than about 1000 Watts of total power, wherein the process is conducted for a time sufficient to modify at least one physical property of a substance within the well, pipeline, or tank while consuming no more than about 1000 Watts of power.

One system of the invention comprises a frequency generator capable of producing frequency radio waves having a frequency of about 1 to about 900 MHz, a RF power amplifier electrically coupled to the radio frequency generator, a microwave frequency generator and microwave amplifier producing microwaves, and a crude stream conduit, wherein each of the frequency generators are disposed proximate to at least a portion of the crude stream conduit, for example, the wellhead of an oil or gas well. In at least one embodiment of the present invention, the system further comprises a low pass filter assembly coupled to the at least one of the amplifiers wherein the low pass filter assembly filters out frequencies produced by the radio and/or microwave frequency generator that may interfere with commercial transmissions. It has been found that this invention has a variety of applications, including, but not limited to, breaking down paraffin buildup within a well bore of an oil or gas well. This and other applications of the invention may be carried out at relatively low power output conditions, as noted above and as will be further described below.

In one particular implementation of the invention, the radio frequency generator comprises four voltage-controlled oscillators (VCO) that are capable of producing a broad range of electromagnetic waves. The spectrum of radio waves produced by this particular frequency generator may include, e.g., ranges of 45-70 MHz, 60-110 MHz, 110-140 MHz, and 140-200 MHz. It should be appreciated, however, that any commercial frequency generator may be used that is capable of producing frequencies within a range of about 1 MHz to about 900 MHz and capable of producing the power output as disclosed below when used in conjunction with the RF power amplifier. In one implementation, the microwave frequencies are generated by a separate microwave generator and amplifier combination powered by a fly-back & Kuk voltage control, wherein a −8V, 3.5V, 5V, and 12V variable source may be used to control the microwave signal. However, it should be appreciated that any commercial microwave generator may be used that is capable of producing frequencies in the range of about 20 GHz to about 40 GHz and capable of producing the power output as disclosed below when used in conjunction with the microwave amplifier. For example, the microwave frequency generator is a conventional type, such as that which is commercially available from Phase Matrix, Inc. of San Jose, Calif. The microwave frequencies generated by the frequency generator in one implementation include ranges of about 19 to about 24 GHz and about 24 to about 30 GHz, wherein these frequencies are generated and amplified with a power output of up to about 1 W. In another implementation, the power output of the microwave amplifier may be up to about 8 W. The output of the very high frequency generator is fed to a RF power amplifier. The RF power amplifier may be any commercially available amplifier capable of producing a power output with a range of about 30 to about 1000 Watts. For example, the RF amplifier may be one commercially available from AR Modular RF of Bothell, Wash. The AR Modular RF unit requires only 110 V_{AC} and produces a maximum of about 40 watts of power for the very high RF frequencies, whereas the microwave amplifier produces about 1 Watt for the microwave frequencies. An example of a radio frequency generator is shown in the attached schematic diagram (consisting of FIGS. 2A, 2B, 2C and 2D).

In another aspect of the invention, a method of altering the composition of hydrocarbons down hole in a well is provided. This method comprises placing the frequency generators electrically coupled to their respective amplifiers as disclosed above proximate to a wellhead in such a manner that the electromagnetic waves produced by the frequency generators may be transmitted into the well; generating a first signal from the radio frequency generator and RF amplifier, the first signal comprising a radio frequency electromagnetic wave; generating a second signal from the microwave frequency generator and amplifier, the second signal comprising a microwave frequency electromagnetic wave; and transmitting the first signal and the second signal into the well, wherein the first signal and the second signal alter the composition of at least one hydrocarbon in the well.

In certain aspects of the invention, the first signal and the second signal may be combined and transmitted into the well simultaneously. The first signal may be a carrier wave for the second signal, which may be the program signal. The signals may be mixed or in certain implementations, the first signal may be transmitted separately from the second signal.

The methods of this invention include generating a radio frequency electromagnetic wave. A radio frequency generator may be used to produce frequencies in the range of about 1 to about 900 MHz, and preferably, the radio frequency electromagnetic wave may be in the frequency ranges of 45-70 MHz, 60-110 MHz, 110-140 MHz, and 140-200 MHz, while most preferably, the radio frequencies may be in the range of about 40 to about 50 MHz. The microwave frequency electromagnetic wave may be in the ranges of about 19 to about 24 GHz and about 24 to about 30 GHz. Without being bound to theory, it is believed that the radio frequency ranges and the microwave frequency ranges may correspond to the quantum spin level of the nucleus and the electron, respectively. It is desirable for each of the spin states energy levels of the nuclear protons and electrons of hydrocarbons found in the well to be found within the ranges of the electromagnetic radiation transmitted.

In another aspect of the present invention, a system for altering the composition of hydrocarbons down hole in a well comprises at least one frequency generator capable of generating radio and microwave frequencies, a crude stream conduit, wherein at least one of the frequency generators is disposed proximate to the crude stream conduit. By proximate it is meant that the generator is sufficiently close to the conduit that the output has the desired effective on at least one hydrocarbon within the well bore. In most cases, the distance of the generator from the conduit will be something less than 2 meters. The crude stream conduit in this embodiment is a well comprising a wellhead assembly, tubing, and casing. The system further comprises an electrical conduit connecting the frequency generator to the tubing located in the well and a wave-guide proximate to the tubing and casing, wherein the waveguide is inserted into an annular space therebetween. The electrical conduit must be a coaxial cable, for example. The well head assembly, tubing, and casing will serve as the transmitting antenna for the 40 to 100 MHz RF signal, while the wave-guide will be the transmitter for the microwave 24-30 GHz signal. In an alternate embodiment, the well head assembly, tubing, and casing will also serve as the transmitting antenna for the microwave signal.

In yet another aspect of the present invention, a method of altering the composition of hydrocarbons down hole in a well comprises placing a transmitting unit (electronic component case) comprising a RF frequency generator and a microwave frequency generator and respective power amplifiers proximate to a crude stream conduit. In this embodiment, the crude stream conduit is a well comprising a wellhead assembly, tubing, and casing. The transmitting unit may include a housing for the frequency generators and respective amplifiers. The method further comprises attaching an electronic conduit to the well head assembly or tubing of the well and placing a wave-guide for the microwave frequency generated electromagnetic waves in the annular space (between the tubing and the casing). The electrical conduit may be a coaxial cable, for example. The tubing and casing will be the transmitting antenna for the 40 to 100 MHz RF, while the wave-guide will be the transmitter for the microwave 24-30 GHz signal. A signal analyzer or oscilloscope may be used to adjust the radio and/or microwave signals to achieve optimal signals. The method further comprises transmitting the radio signal and the microwave signal into the well, wherein the radio signal and the microwave signal alter the composition of at least one hydrocarbon in the well. The transmitting unit may operate continuously or intermittently. In certain embodiments of the invention, it will operate continuously at first for a period of time (e.g., in the range of 100 to 1000 hours), but later be set to an intermittent mode (e.g., pulsing every 1800 to 3600 seconds). The duration of operation may be more or less than these durations, and will vary depending upon production volumes, the desired effect and the magnitude of the problem confronted (blockage down hole, for example).

These and other embodiments, features and advantages of the present invention will be further evident from the ensuing detailed description, including the appended figures and claims.

### SUMMARY OF THE FIGURES

FIG. 1 is a graphical representation of data obtained from the GC and MS analysis of Gulf wax diluted in diesel samples before and after treatment in accordance with the present invention, with an overlay graph showing the difference, in area percent, for each carbon chain length present in the sample after treatment in accordance with the invention.

FIGS. 2A, 2B, 2C and 2D, together, are a schematic diagram of the circuitry of a frequency generator of one embodiment of the present invention.

FIGS. 3A and 3B are a graphical representation of data obtained from the GC and MS analysis of docosane diluted in diesel samples before and after treatment in accordance with the present invention, showing the difference, in area percent, for each carbon chain length present in the sample before and after treatment in accordance with the invention.

FIG. 4 is a graphical representation of data obtained from the gas chromatography analysis of a Well #174 before and after treatment in accordance with the present invention, showing the difference, in area percent by gas chromatography, for the percentage of higher carbon fractions produced.

FIG. 5 is a block diagram of one embodiment of the present invention of the system used to transmit radio and/or microwave transmissions to hydrocarbonaceous material. The block diagram includes the signal generating unit, the amplifier, the SWR meter, the impedance matching network, and the dipole antenna or well head assembly.

FIG. 6 is a Summary of Effective Permeability Results as disclosed in Example 8.

FIG. 7 is a group of scanning electron micrograph images of calcium sulfate samples described in Example 9.

FIG. 8 is a group of scanning electron micrograph images of barium sulfate samples described in Example 9.

Like reference indicators are used to refer to like parts or steps described amongst the several figures.

### FURTHER DETAILED DESCRIPTION OF THE INVENTION

Without being bound by theory, it is believed that this invention takes advantage of the spin properties of atoms and molecules. Proton or hydrogen spin state (l=½) is perturbed by electromagnetic radiation in the 3 to 100 MHz range (NMR or Nuclear Magnetic Resonance), and electron spin is perturbed by electromagnetic waves in the 24 to 30 GHz range (ESR or Electron Spin Resonance). If the energy supplied by the radiation is sufficient to alter the spin states of one or both the proton and the electron then the promoted spin states of each will act to accommodate or discourage hydrogen bonding or cleavage. In addition to bonding, radicals formed in the process of going from the ground state to an elevated energy state are capable of abstracting hydrogen from carbon chains and leaving a point of attack in the molecule. If the attack takes place on adjacent carbons double bonds can result, but the attacks do not stop at this stage; they go on and carbon-carbon bond cleavage can result. This can take place even if the radiation is of very low energy (e.g., 31 total Watts) with the process of cleaving and isomerization occurring because of quantum tunneling. This then means that although carbon-carbon bond cleavage is energetically unfavorable under the conditions of low power irradiation (from 30 to 300 Watts), it can still take place because of the enormous incidence of wave particle interaction under the conditions of this invention.

In one embodiment of the present invention, a process is provided to expose a substance to electromagnetic waves and to detectably alter at least one physical property of the substance as it existed prior to the exposure. Substances to be altered will include hydrocarbonaceous material and will generally include hydrocarbons associated with oil and gas production and their location within well bores, formations, pipelines, storage tanks, and the like. The process includes providing a radio frequency generator capable of producing radio frequencies in the range of about 1 MHz to about 900 MHz. It should be appreciated that the radio frequency generator may be any commercially available frequency generator capable of producing the frequencies in the above mentioned range. Preferably, the radio frequency generator may generate electromagnetic waves having a frequency of about 1 MHz to about 100 MHz, Still more preferable, the radio frequency generator may generate electromagnetic waves having a frequency of about 30 MHz to about 50 MHz. Still yet more preferable, the radio frequency generator may generate electromagnetic waves having a frequency of about 40 MHz to about 50 MHz. Most preferably, the radio frequency generator may generate electromagnetic waves having a frequency of at least about 46.2 MHz.

In one embodiment, a radio frequency power amplifier is electrically coupled to the radio frequency generator. The radio frequency power amplifier may be any RF power amplifier capable of receiving the signal from the frequency generator, wherein the signal has a frequency in the range of about 1 MHz to about 900 MHz, and further capable of producing a power output of about 30 W to about 1000 W. It should be appreciated that the frequency generator and amplifier may be separate components or may be constructed so as to form an integral unit. The radio frequency generator and RF power amplifier in combination generate and amplify electromagnetic waves at a selected frequency in the range of the frequencies mentioned above. It should be appreciated that the frequency generator and amplifier may be powered by a generator or other means depending on the environment in which the hydrocarbonaceous material is found, e.g., a well site, pipeline facility, refinery, etc. Other electrical components such as, for example, a AC/DC converter or duty cycle timer may be used. The radio frequency generator and RF amplifier and other electrical components, including a microwave generator and amplifier discussed below, may be contained in a housing or transmittal unit.

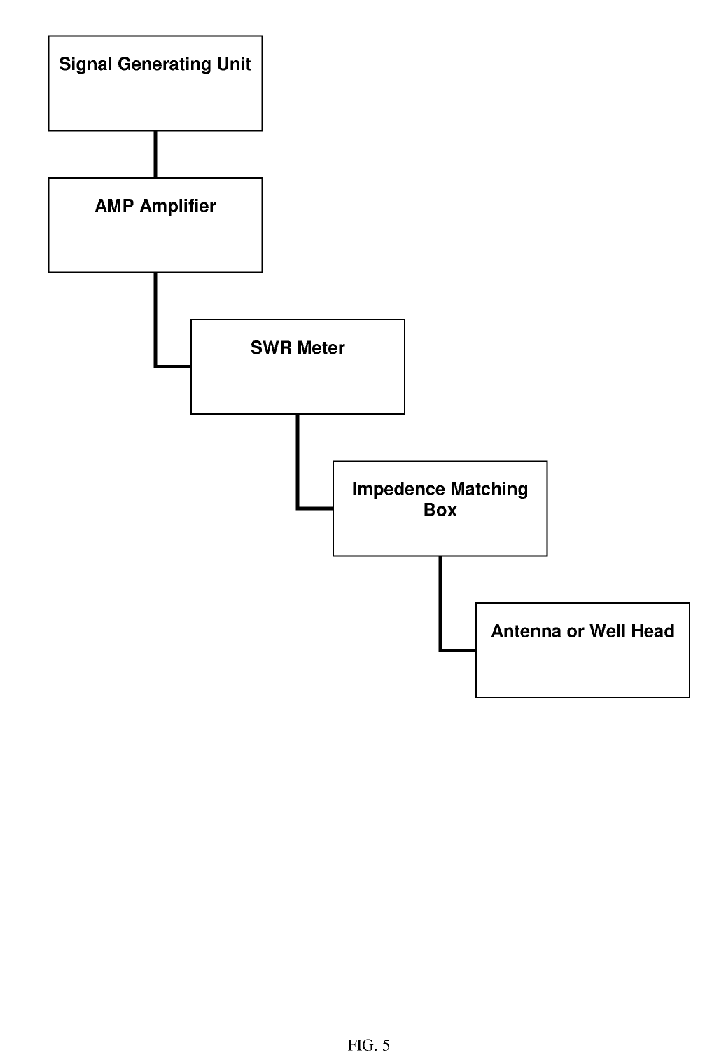

The RF amplifier may be electrically coupled to a standing wave ratio (SWR) meter, wherein the SWR meter is electrically coupled to an impedance matching network in at least one embodiment of the present invention. The SWR meter may be used to measure the forward power versus the reflected power. The SWR meter is indicative of the impedance match between the radio frequency generator and amplifier, i.e., signal generating unit, and the load impedance, which will be discussed further below. The impedance matching network will be electrically coupled to a transmitting device or antenna. It should be appreciated that in certain embodiments, the SWR meter and the impedance matching network may be an integral unit. For example, the integral unit may be a MAC-200, manufactured by SGC of Bellevue, Wash. FIG. 5 illustrates a block diagram of the configuration in one embodiment of the present invention.

The antenna used in one embodiment may be the well head assembly, tubing, and casing of an oil or gas well. In such an embodiment, the impedance matching network is electrically coupled to the well head assembly, casing, and tubing. One end of a coaxial cable is coupled to the impedance matching network and the other end of the coaxial cable will be electrically coupled to the well head assembly, casing, and tubing. Specifically, the braided outer conductor of the coaxial cable will be attached to a metal stake placed in the surface of the earth proximate to the well to serve as the ground. The center wire of the coaxial cable will be coupled to the well head assembly, typically the flow line of the well. As such, the entire well head assembly, casing, and tubing is conductive and serves as the antenna.

In another embodiment, the antenna may be at least one dipole antenna. In another embodiment, the antenna may be at least one monopole antenna. In certain embodiments, the dipole antenna may be a quarter wave or half wave dipole antenna. The dipole antenna may be coupled to the impedance matching network by coaxial cable and run into the well head assembly through the gate valve in the well head assembly. In such an embodiment, the dipole antenna will be disposed within the annulus of a well bore comprising casing and tubing. The length of the dipole antenna will vary based on its characteristics, e.g., half wave, full wave, etc. In one embodiment, the dipole antenna is disposed at a depth of about twelve feet (3.66 meters) from the well head assembly in the annulus. It should be appreciated that the antenna may also be run through the tubing in certain embodiments.

Additionally, the monopole or dipole antenna may be disposed within a pipeline or tank comprising hydrocarbonaceous material. In one embodiment, a dipole antenna is inserted into one end of the pipeline, approximately eight feet (2.44 meters) to twelve feet (3.66 meters) into an inner central portion of the end portion of the pipeline. In another embodiment, a dipole or monopole antenna is inserted into each end portion of the pipeline. In still yet another embodiment, a monopole or dipole antenna may be inserted into a tank comprising hydrocarbonaceous material. In the embodiments disclosed above, the dipole or monopole antennas may transmit radio waves and/or microwaves. In certain embodiments, radio and microwaves may be transmitted on a single antenna. In at least one embodiment, radio waves will be transmitted on a separate antenna from the antenna transmitting microwaves.

Optionally, a microwave frequency generator may be provided, the microwave generator being any commercially available microwave generator capable of producing electromagnetic waves having a frequency range of about 20 to about 40 GHz. Preferably, the microwave frequency generator produces electromagnetic waves having a frequency range of about 20 GHz to about 30 GHz. Most preferably, the microwave frequency generator produces electromagnetic waves having a frequency range of at least about 24 GHz. In one embodiment, the microwave generator is electrically coupled to a microwave amplifier, the amplifier being any commercially available amplifier capable of receiving the signal from the microwave frequency generator, wherein the signal has a frequency in the range of about 20 GHz to about 40 GHz, and further capable of producing a power output of up to about 8 W. It should be appreciated that the frequency generator and amplifier may be separate components or may be constructed so as to form an integral unit. In at least one embodiment, the radio frequency generator and RF amplifier and the microwave frequency generator and amplifier are all housed in a single transmittal unit. Microwaves may be transmitted in conjunction with the radio waves, and may be transmitted concurrently or before or after the radio waves are transmitted.

In one embodiment, the microwave amplifier is electrically coupled to the antenna. The antenna may be a dipole antenna, a monopole antenna, or the well head assembly, tubing, and casing disclosed above. The microwaves and radio waves may be transmitted from a single antenna or each amplifier may be electrically coupled to a separate antenna. In coupling the microwave amplifier to the antenna, a coaxial cable is used. One end of the coaxial cable is coupled to the microwave amplifier whereas the other end of the coaxial cable is coupled to the dipole antenna. In another embodiment, the antenna is the well head assembly, tubing, and casing. In such an embodiment, the end of the coaxial cable not coupled to the microwave amplifier is coupled to the well head assembly, wherein the center wire of the coaxial cable is attached to the polished rod of the well head assembly and the outer sheath of the coaxial cable is attached to a metal stake urged into the surface of the earth, thus functioning as a ground wire.

The impedance matching network will function to match the output impedance of the signal generating unit, wherein the signal generating unit comprises the radio frequency generator and RF amplifier, with the load impedance, wherein the load impedance may be defined as the impedance of the antenna and the coaxial cable coupling the antenna to the impedance matching network. The impedance matching network may be adjusted manually or automatically. In adjusting the impedance matching network, the impedance matching network comprises variable inductors and variable capacitors capable of varying the impedance in order to match the output impedance of the signal generating unit with the load impedance. The impedance may be matched automatically by the use of such devices as the MAC-200 disclosed above. It should be appreciated that the foregoing system to transmit the electromagnetic waves generated by a radio frequency generator and the microwave frequency generator consumes no more than about 1,000 Watts of power

### EXAMPLE 1

The foregoing has been confirmed by Gas Chromatography combined with Mass Spectroscopy used to examine a sample of Gulf wax (food grade) diluted with xylene (27% by weight) before and after irradiation. Treatment was made by exposing samples to be treated to radio frequency (76 MHz) electromagnetic waves and microwaves (29 GHz) for a period of 2.5 hours. Aliquots of 25 ml were taken from the very bottom of the graduated cylinders treated and untreated samples and placed in two weigh dishes. The samples were then placed in a room temperature (25° C.) vacuum oven and a 22 inch vacuum was pulled on the samples until they contained no more solvent. After the samples had lost all their solvent the weigh dishes were weighed to compare the amount of material in each. The treated sample was found to contain 20% less by weight than the untreated sample, verifying that the RF/Microwave treatment kept more of the wax in solution than the untreated sample.

### EXAMPLE 2

Gulf wax (food grade) similarly diluted in diesel was further analyzed before and after RF/Microwave treatment. Results are summarized in Table 1 below.

TABLE 1

| Total Gulf Wax Charged grams | Total Diesel grams |
|---|---|
| 235.00 | 870.00 |
| Wt % Wax | Wt % Diesel |
| 21.27 | 78.73 |
| Percent Wax recovered by | Percent Wax recovered by |
| filtration (after RF treatment) | filtration (no treatment) |
| 40.63 | 93.54 |
| Percent Wax left in Diesel | Percent Wax left in Diesel |
| (treated) | (no treatment) |
| 59.37 | 6.46 |

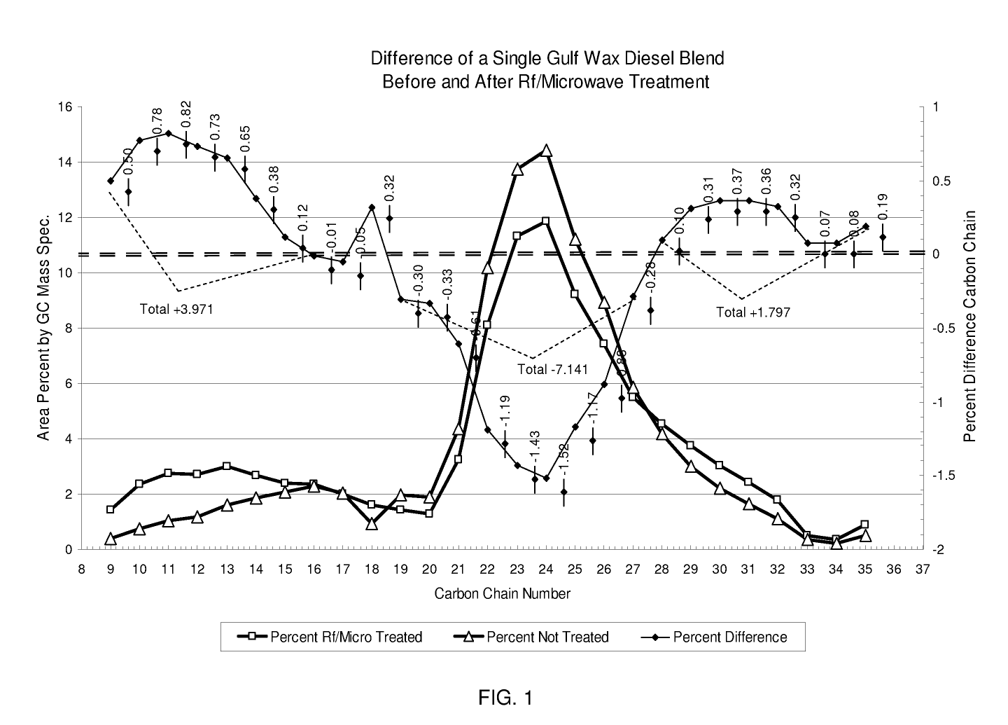

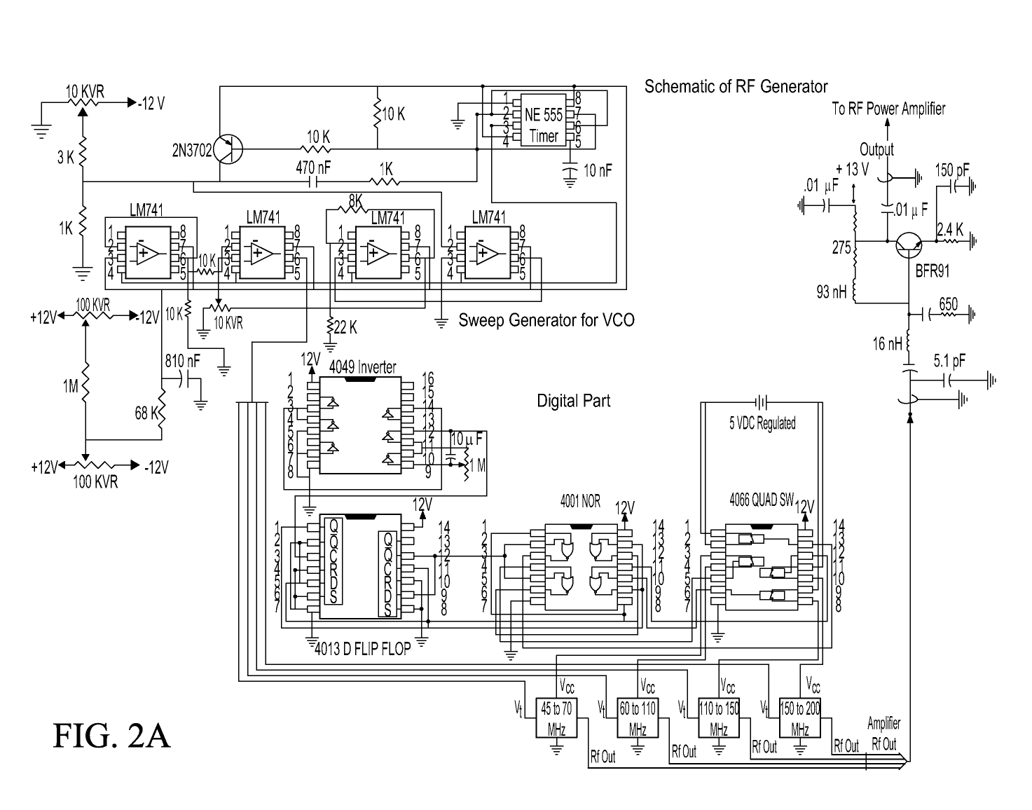

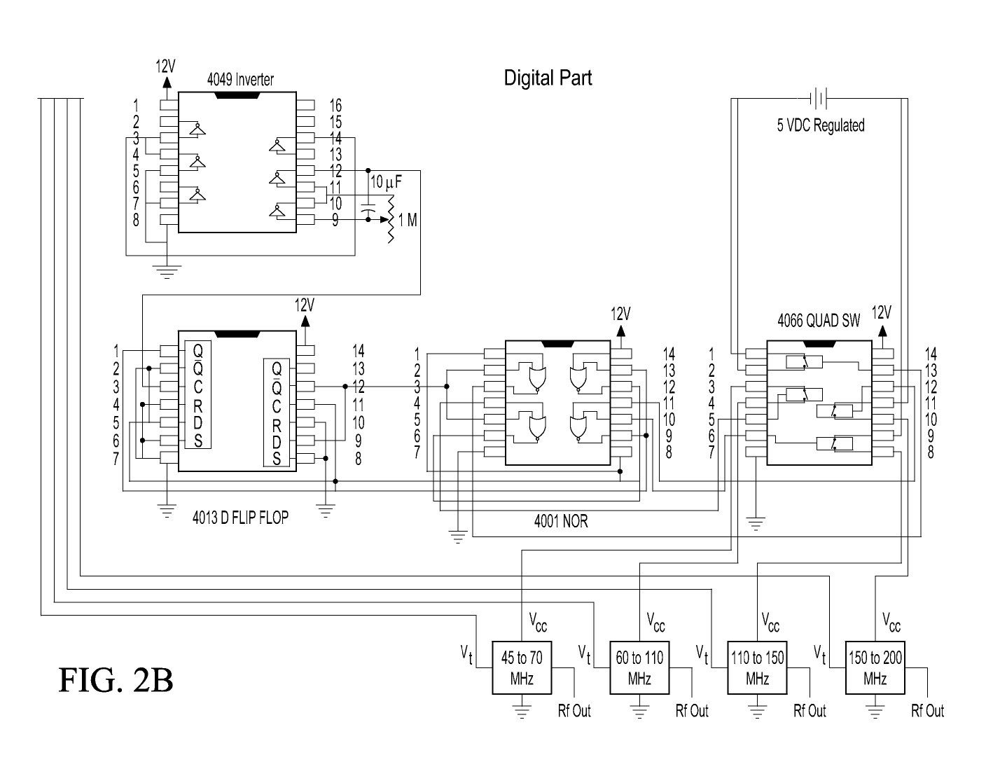

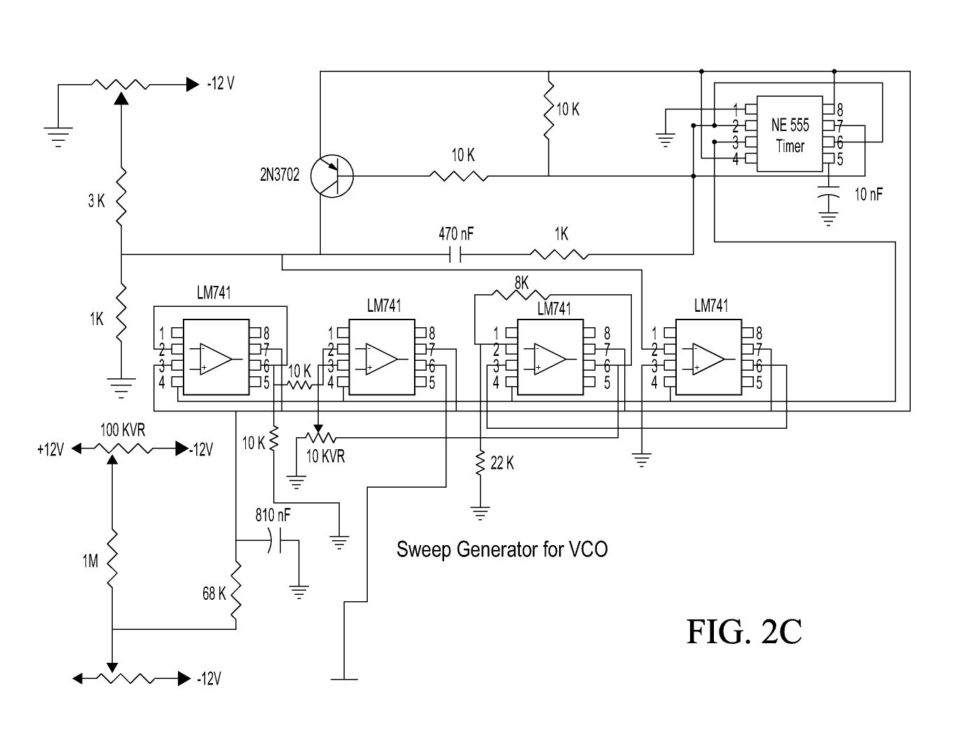

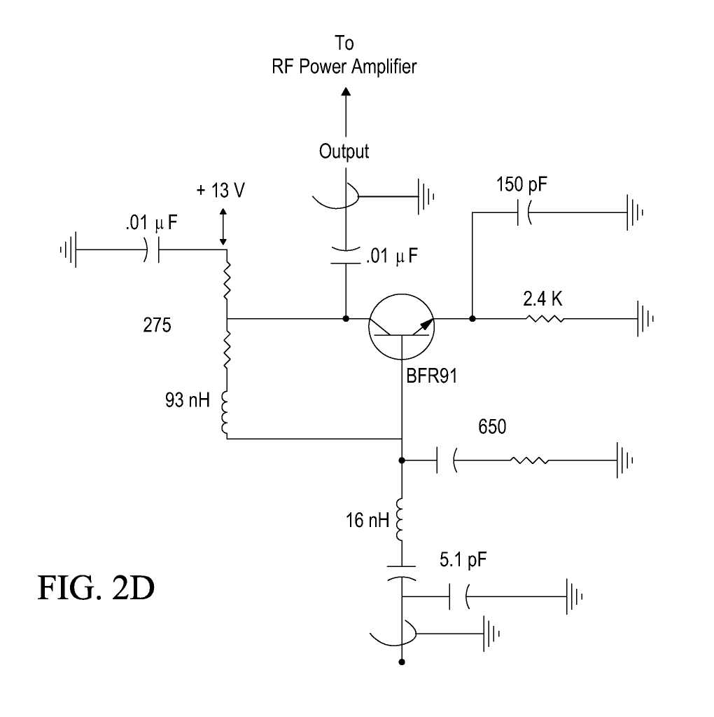

Gas Chromatography and Mass Spectrometry analysis revealed that the RF/Microwave treated sample gave a larger percentage of lower carbon number species, a clear decrease in the waxy carbon 18 to 30 chain lengths, and an increase in some 30+ carbon chains, all of which is quite consistent with carbon-carbon bond breakdown seen in other methods of hydrocarbon cracking. FIG. 1 graphically illustrates the data obtained.

### EXAMPLE 3

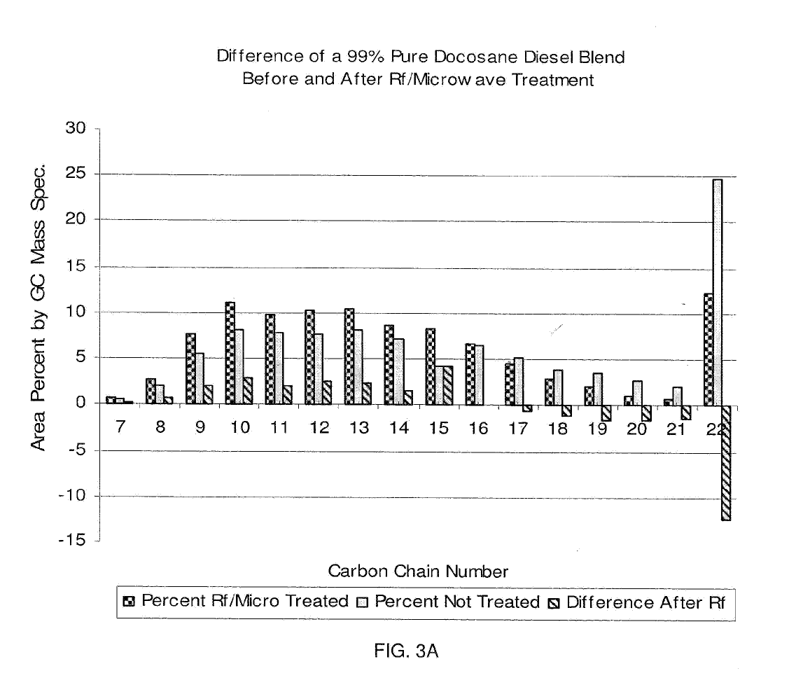

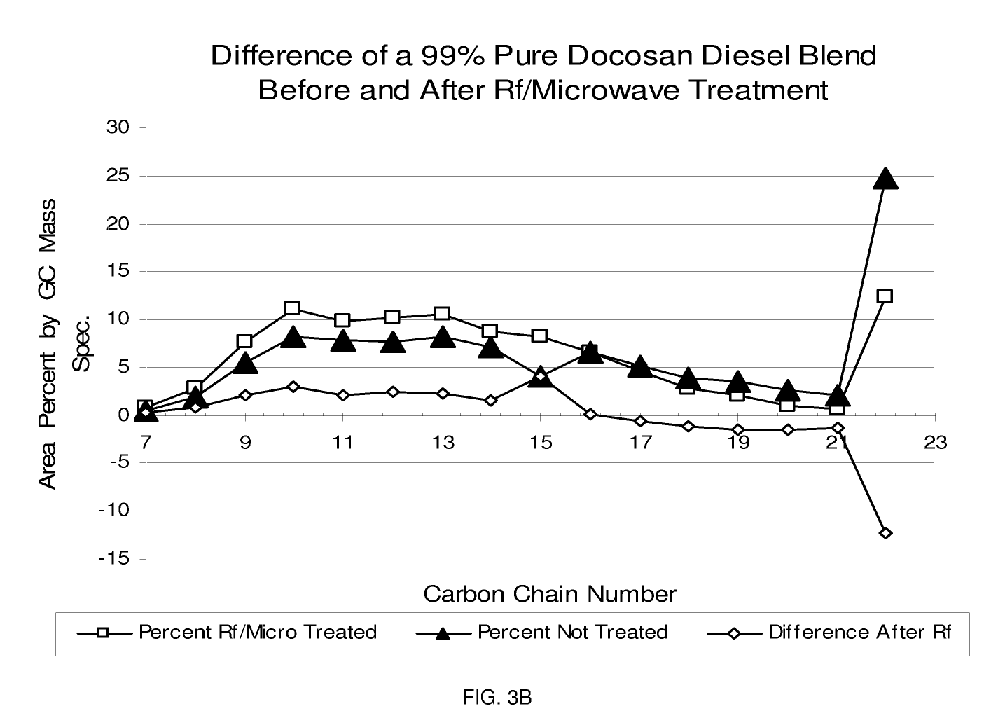

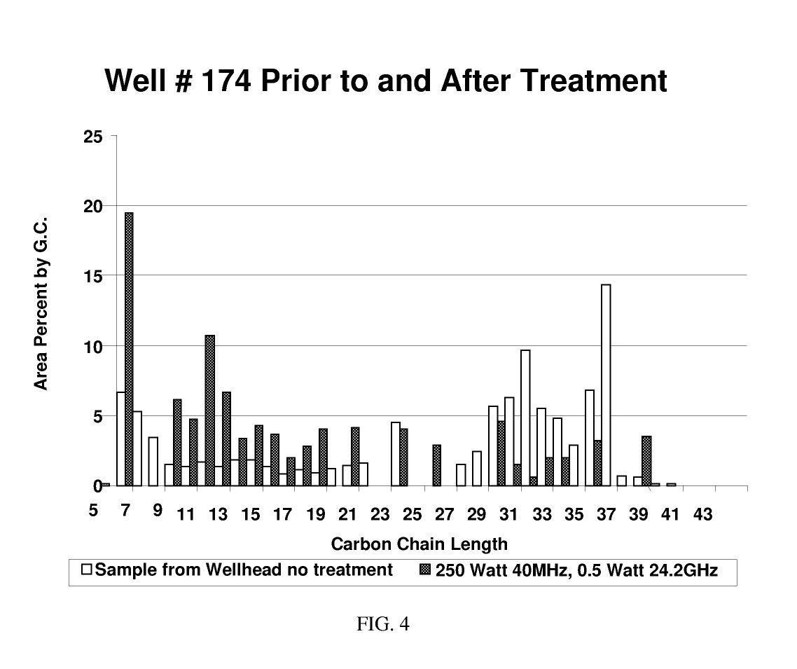

The procedure of Example 2 was repeated, except that Aldrich reagent grade, 99 percent pure docosane was substituted for the Gulf wax of Example 2. The resulting Gas Chromatography/Mass Spectrometry analysis is plotted on FIGS. 3A and 3B. It is apparent that the results do not show clear cut indications of carbon-carbon cleavage. It appears likely that the two electromagnetic wave frequencies interact with forming hydrogen bonds to prevent aggregation of the wax crystals to form wax deposits.

### EXAMPLE 4

At least one method as disclosed above was applied to seventeen oil wells located in West Texas, wherein radio (40.68 MHz) at 40 Watts and microwave (24.4 GHz) at 1 Watt signals were transmitted into the well bore by a transmitting unit. All seventeen wells were observed to have positive effects (e.g., increased oil production, increased total fluid, solid paraffin removal, flow line pressure drops, and added gas production) upon exposure to the radio and microwave signals. The combination frequency effects have proven to affect intermolecular aggregation, and anecdotal evidence has confirmed these frequencies are effective in removing near well bore damage. Results of this experiment are summarized in Table 2.

TABLE 2

| Well | Bbls Oil | Bbls Water | Bbls Oil | Bbls Water | |
|---|---|---|---|---|---|
| No. | before RF | before RF | after RF | after RF | Comments |
| 348 | 2 | 15 | 16 | 107 | Lots of gas |
| 336 | 8 | 80 | 10 | 56 | Lots of gas |
| 527 | 8 | 112 | 9 | 112 | Lots of gas |
| | | | | | and water |
| 394 | 3 | 10 | 8 | 9 | Lots of gas |
| 493 | 12 | 34 | 15 | 29 | Lots of gas |
| 550 | 9 | 20 | 11 | 13 | Big wads |
| | | | | | wax released |
| 498 | 15 | 20 | 17 | 20 | Lots of gas |
| 365 | 9 | 22 | 12 | 20 | Lots of gas |
| 91 | 10 | 30 | 13 | 40 | Lots of gas |
| 538 | 9 | 50 | 11 | 65 | Lots of gas |
| 31 | 7 | 8 | 11 | 8 | Lots of gas |
| 27 | 6 | 11 | 9 | 12 | Lots of gas |
| 375 | 8 | 21 | 11 | 14 | Lots of gas |
| 438 | 8 | 44 | 12 | 53 | |
| 398 | 4 | 18 | 7 | 19 | Lots of gas |
| 174 | 3 | 22 | 25 | 12 | Lots of gas |
| Quan- | 2 | 29 | 12 | 35 | Lots of gas |
| tum | | | | | |
| Total | 123 | | 210 | Increase | 87 |
| | | | | Bbl. Oil | |

### EXAMPLE 5

Well testing by oil company personnel was performed after the treatments as disclosed above on these five oil wells located in West Texas for an extended period of time, the period of time lasting for at least two weeks and summarized in Table 3 below. Radio waves (40.68 MHz) at 40 Watts and microwave waves (24.4 GHz) at 1 Watt signals were transmitted into the well bore by a transmitting unit at time intervals of no more than two hours. All five wells were observed to have positive effects (e.g., increased oil production, increased total fluid, solid paraffin removal, flow line pressure drops, and added gas production) upon exposure to the radio and microwave signals. The combination frequency effects have proven to affect intermolecular aggregation, and anecdotal evidence has confirmed these frequencies are effective in removing near well bore damage. Results of this experiment are summarized in Table 3.

TABLE 3

| Well | Bbls Oil | Bbls Water | Bbls Oil | Bbls Water | |
|---|---|---|---|---|---|
| No. | before RF | before RF | after RF | after RF | Comments |
| 348 | 12 | 22 | 17 | 56 | Lots of gas |
| | | | | | Test lasted 2 |
| | | | | | weeks |
| 336 | 6 | 77 | 11 | 53 | Lots of gas |
| | | | | | Test lasted 2 |
| | | | | | weeks |
| 498 | 17 | 22 | 23 | 27 | Lots of gas |
| | | | | | Test lasted 3 |
| | | | | | weeks |
| 438 | 12 | 48 | 16 | 56 | Lots of gas |
| | | | | | Test lasted 2 |
| | | | | | weeks |
| 174 | 9 | 5 | 14 | 9 | Lots of gas |
| | | | | | Test lasted 2 |
| | | | | | weeks |
| Total | 56 | | 81 | Increase | 25 |
| | | | | Bbls. Oil | |

### EXAMPLE 6

Initially, a well was plugged off with paraffin wax and the operating company could not pump any solvent into the well. The well was treated with radio signals and microwave signals of 40 MHz and 24 GHz, respectively. After an hour, the tubing pressure rose to 1,000 psi (68.95 bar). An attempt to flow the well was made, but the differential pressure was too great. After opening the flow line, the pressure dropped back to 0 psi (0 bar) and it took another 20 minutes to gain 1,000 psi (68.95 bar). The flow line was opened again and the pressure dropped to 0 psi (0 bar) again. The tubing pressure was increased to 1,500 psi (103.42 bar). A subsequent operator observed that the wax obstruction was removed down to 750 feet (228.60 meters). It appears the exposure of the paraffin wax to the radio waves and microwaves resulted in a decrease in the obstruction of the paraffin wax in the well.

### EXAMPLE 7

Three wells were treated with the same RF and microwave frequency set up, except that power for the VHF RF transmitter was 50 Watts and the transmitters were connected to two antennae, and those were inserted twelve (12) feet (3.66 meters) into the back side annular space of a low-pressure well that had its pressure bled off prior to antennae placement. The unit was powered up and remained on for two (2) hours. Two days later, well test was run on each well, and production increase was 5 bbls. oil increase per day on two of the wells, and 3 bbls. oil increase in production on the third.

### EXAMPLE 8

Formation material from natively oil-wet sandstone was used in this study. Cylindrical test samples were drilled using Isopar-L as the bit coolant and lubricant. The samples were trimmed to right cylinders prior to use. Mineralogical information had previously been determined and is listed below.

TABLE 4
Summary of X-Ray Diffraction (wt. %)

| Mineral Phases | (wt. %) |
|---|---|
| Quartz | 62 |
| Plagioclase Feldspar | 8 |
| Potassium Feldspar | 10 |
| Dolomite | 1 |
| Kaolinite | 4 |
| Mica and/or Illite | 2 |
| Mixed-Layer Illite₉₀/Smectite₁₀ | 12 |

### Flow Test Conditions:

Temperature: 150° F. (65.56° C.)
Net Confining Stress: 1500 psi (103.42 bar)
Backpressure=200 psi (13.79 bar)

### Fluids:

Brine: Two percent by weight potassium chloride (2% KCl) solution, prepared with deionized water and reagent grade salts. Filtered and evacuated prior to use.
Crude Oil: Heavy crude oil known to contain asphaltenes. Viscosity at test temperature=16.2 centipoise (cp).
Mineral Oil: Isopar-L, a laboratory grade mineral oil. Filtered and evacuated prior to use. Viscosity at test temperature=0.96 cp.

### Flow Test Procedures:

### Effective Permeability to Water at Residual Oil Saturation, KwSor (Native-State Condition)

The sample was loaded under confining stress in a HASSLER load coreholder. The 2% KCl brine was injected against 200 psi (13.79 bar) backpressure at a constant flow rate. Differential pressure was monitored and an effective permeability to water at residual oil (KwSor) is calculated. KwSor=3.04 mD (millidarcies)

### Effective Permeability to Oil at Irreducible Water Saturation, KoSwi

Heavy crude oil injection against 200 psi (13.79 bar) backpressure followed brine injection to establish irreducible water saturation and to potentially place asphaltenes on the grain surfaces. Differential pressure and flow rate were monitored and an effective permeability to oil at irreducible water saturation (KoSwi) was calculated. Crude Oil KoSwi=0.890 mD.

Isopar-L was injected against 200 psi (13.79 bar) backpressure to remove the crude oil from the pore space. Differential pressure and flow rate were monitored to allow calculation of KoSwi prior to RF treatment. KoSwi=0.937 mD.

### RF Treatment

The coreholder assembly with the test sample still loaded, was transported for RF treatment and returned. The RF treatment was carried out as follows: Core sample was placed inside the rubber bladder of a Hassler-type core holder between the two feed lines of the end plates. The RF transmission line ground (outer shield of the coaxial cable) was place on one end feed line and the center of the coaxial cable was attached to the other feed line. The microwave transmission line was wrapped around the rubber bladder (which is permeable to both RF and microwave). 50 watts of RF at 40 MHz and 1 watt of microwave at 24 GHz was applied for approximately 7.5 minutes. Power was then turned off and the sample was ready for analysis.

Effective Permeability to Oil at Irreducible Water Saturation, KoSwi Post Treatment

Following RF treatment, Isopar-L was injected against 200 psi (13.79 bar) backpressure. Differential pressure and flow rate were monitored to allow calculation of KoSwi after RF treatment. KoSwi after treatment=1.80 mD, indicating a significant improvement in oil productivity.

### Effective Permeability to Water at Residual Oil Saturation, KwSor Post Treatment

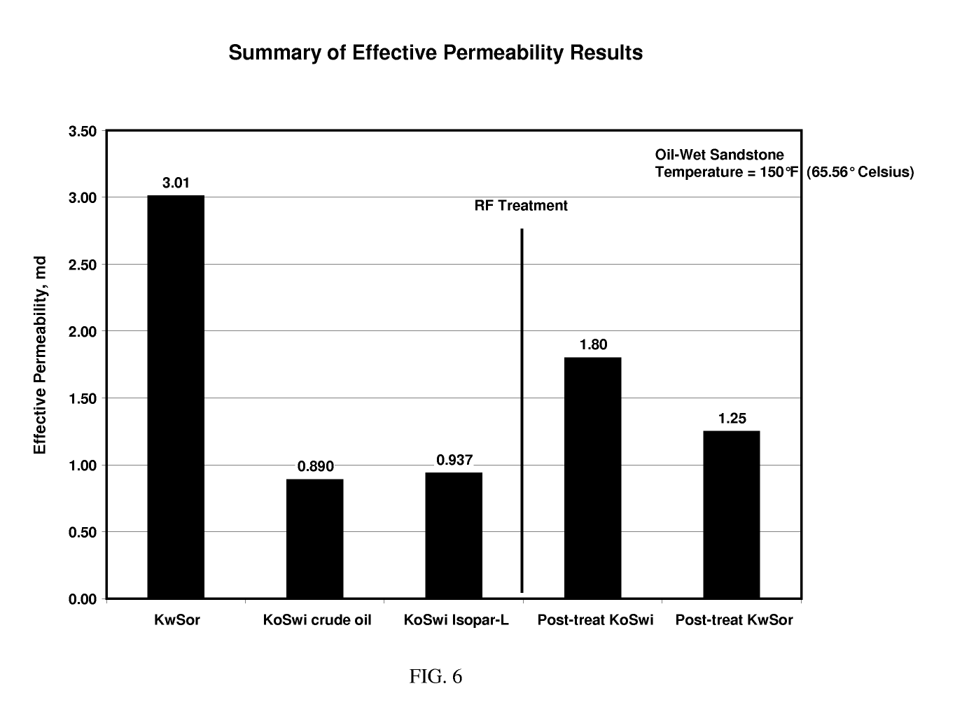

The 2% KCl brine was injected against 200 psi (13.79 bar) backpressure at a constant flow rate to establish residual oil saturation. Differential pressure was monitored and KwSor after treatment was calculated as 1.25 mD, a decline in water productivity exceeding 50%. A summary of effective permeability results is illustrated in the graph found in FIG. 6. From the numbers presented in FIG. 6, it can be seen that the ratio of hydrocarbon effective permeability (e.g., crude oil) to water effective permeability (the oil to water mobility ratio) increased from 0.3 prior to treatment to 1.44 after treatment. This represents a substantial increase in the permeability of hydrocarbon and concurrent substantial decrease in the permeability of water in the formation sample which underwent treatment.

### EXAMPLE 9

Two sets of two aqueous solutions were formed. The first set included a solution of calcium chloride (5-20 wt % based on the weight of the solution) in distilled water, and a solution of sodium bicarbonate (5-20 wt % based on the weight of the solution) in distilled water, which when mixed together form calcium carbonate scale. The second set included a solution of barium chloride (5-20 wt %) in distilled water, and a solution of sodium sulfate (5-20 wt % based on the weight of the solution) in distilled water which when mixed together forms barium sulfate scale.

For each set, one mixture was exposed to a VHF frequency, from a transmitter using 50 Watts or less, during formation and another mixture was not exposed during formation. This was repeated for different aliquots at different VHF frequencies. Crystallization began shortly after the solutions were brought into contact with one another. Once the crystallization had taken place, where applicable VHF exposure was terminated and the precipitated crystals were filtered and submitted for electron scanning photo-microscopy (ESM). The exposure duration was on average around 2 hours for those aliquots exposed to VHF.

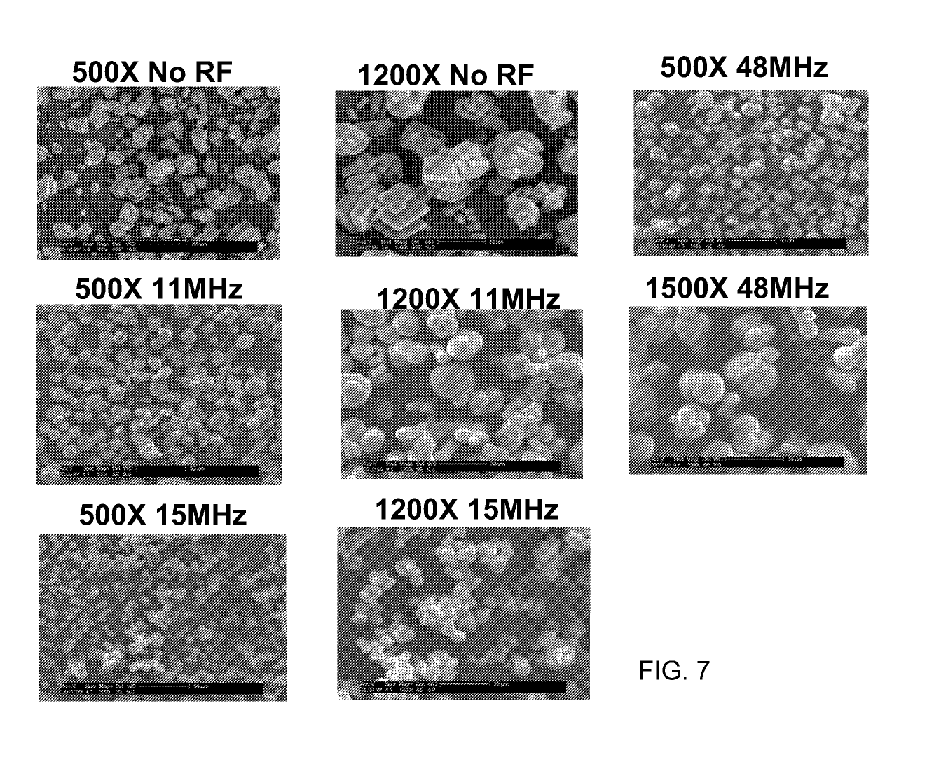

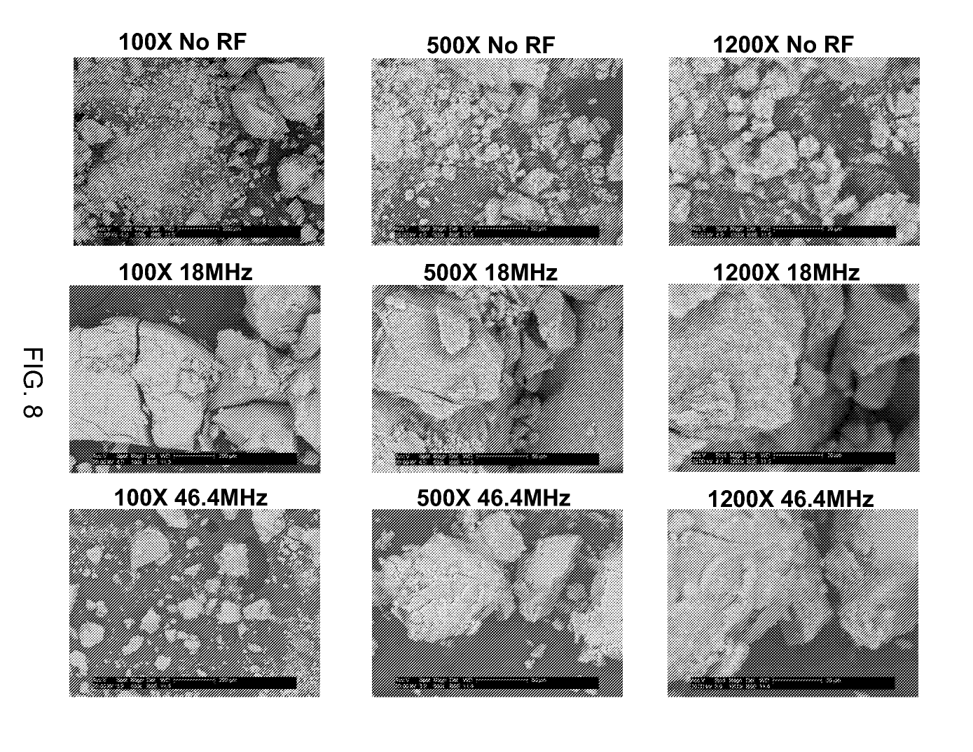

The resulting photo-micrographs and their associated VHF frequencies appear on the slides at FIGS. 7 (for the calcium carbonate samples) and 8 (for the barium sulfate samples). Above each ESM micrograph the magnification and RF treatment frequency, if any, is indicated.

The calcium carbonate gave very good crystals which could be distinguished from the photo-micrographs, while the barium sulfate crystals were amorphous and showed little evidence of morphological changes. Because the micrographs for the barium sulfate crystals were unremarkable, the barium sulfate scale samples were filtered from the solution and timed to determine the tendency to deposit upon filtration. The time to complete filtration was measured. The barium sulfate sample which was not VHF treated and the barium sulfate sample which was treated at 46.4 MHz both took 1.5 hours to filter, while the barium sulfate sample which was VHF treated at 18 MHz took 30 seconds to filter. The latter observation indicated that the crystals of the barium sulfate sample treated at 18 MHz were drier, not as voluminous and tended less to agglomerate, presumably because of a lack of water of hydration.

### EXAMPLE 10

A monopole antenna was placed in the annulus of a well having scale problems associated with its electric submersible pump. The frequency generator was activated at 30 watts of VHF signal at 18 MHz for 1 hour, and finished with 250 Watts of 40 MHz for an additional hour.

The well fluids were sampled before and after the VHF treatment, and the results of the lab analysis are on the following tables.

TABLE 5

| | Saturation | Momentary Excess |
|---|---|---|
| Mineral Scale | Index | (lbs/1000 bbls) |
| Calcite (CaCO3) | 1.17 | 0.01 |
| Strontianite (SrCO3) | 0.03 | −2.18 |
| Anhydrite (CaSO4) | 0.78 | −153.54 |
| Gypsum | 0.98 | −10.53 |
| (CaSO4*2H2O) | 0.45 | −0.59 |
| Barite (BaSO4) | 0.31 | −443.89 |
| Celestite (SrSO4) | 0.06 | −0.47 |
| Siderite (FeCO3) | 0.03 | −438226.56 |
| Halite (NaCl) | 0.72 | −0.04 |
| Iron sulfide (FeS) | — | — |

TABLE 6

| | Saturation | Momentary Excess |
|---|---|---|
| Mineral Scale | Index | (lbs/1000 bbls) |
| Calcite (CaCO3) | 1.73 | 0.02 |
| Strontianite (SrCO3) | 0.03 | −1.63 |
| Anhydrite (CaSO4) | 0.97 | −12.94 |
| Gypsum | 1.17 | 73.72 |
| (CaSO4*2H2O) | 0.08 | −1.50 |
| Barite (BaSO4) | 0.31 | −502.37 |
| Celestite (SrSO4) | 1.78 | 0.02 |
| Siderite (FeCO3) | 0.06 | −378650.75 |
| Halite (NaCl) | 64.45 | 3.59 |
| Iron sulfide (FeS) | — | — |

### EXAMPLE 11

Using the test procedures specified in API Spec. 10A and API RP (Recommended practices) 10B, a single batch of Portland cement was formed and split into four equal portions (of approximately 8 oz. each) in a plastic mold. Two of these portions were exposed to VHF radio waves from a monopole antenna disposed in the cement molds in contact with the wet cement and transmitter at a frequency of 18 MHz for 10 hours while setting at room temperature and pressure, using a power level of 20 to 50 watts. The other two portions set for the same period of time and under the same conditions, except that they were left unexposed to the radio waves. After set up, the two treated and two untreated samples were subjected to a compressive strength test in accord with the above-referenced API test procedures. The results are listed in the following table 7.

TABLE 7

| Sample | Specimen | Width | Thickness | Max Force | Strength |
|---|---|---|---|---|---|
| Number | ID | (in) | (in) | (lbs) | (psi) |
| 3834 | RF treated | 2.05 | 1.97 | 3,737 | 925 |
| 3835 | No treatment | 2.04 | 2.025 | 2,757 | 667 |
| 3836 | No Treatment | 2.06 | 1.96 | 2,907 | 713 |
| 3837 | RF treated | 2.05 | 2.05 | 3,886 | 925 |
| | Mean | 2.055 | 2.006 | 3,322 | 808 |
| | Median | 2.05 | 1.98 | 2,907 | |
| | Std Dev | 0.008 | 0.036 | 572 | 137 |
| | Maximum | 2.06 | 2.05 | 3,886 | 925 |
| | Minimum | 2.04 | 1.97 | 2,757 | 667 |
| | Range | .02 | .08 | 1,2129 | 258 |

### EXAMPLE 12

Samples were prepared as in Example 11, except that each of the sample-containing plastic molds were topped with a covering layer of room-temperature water, with each sample and water-containing mold also being itself immersed in a water bath within a larger plastic tank. The ground wire was placed in the water bath, and the molds-containing larger plastic tank was wrapped with a monopole antenna connected to a radio wave transmitter. The samples were then irradiated for 20 hours at 18 MHz, using a power level of 40 watts.

Upon testing for compressive strength in accordance with the procedures cited in Example 11, it was found that samples irradiated with 18 MHz gave an average compression strength of 1468 psi, while those not irradiated gave an average of 851 psi. The test results are set forth in Tables 8 and 9 below.

TABLE 8

| Sample | Specimen | Width | Thickness | Max Force | Strength |
|---|---|---|---|---|---|
| Number | ID | (in) | (in) | (lbs) | (psi) |
| 3919 | NO RF | 2.015 | 2.05 | 3,746 | 907 |
| 3920 | NO RF | 1.98 | 2.05 | 3,331 | 821 |
| 3921 | NO RF | 2.025 | 2.05 | 3,730 | 898 |
| 3922 | NO RF | 1.980 | 2.04 | 3,143 | 778 |
| | Mean | 2.000 | 2.047 | 3,487 | 851 |
| | Median | 1.98 | 2.05 | 3,331 | |
| | Std Dev | 0.023 | 0.005 | 266 | 62 |
| | Maximum | 2.05 | 2.05 | 3,748 | 907 |
| | Minimum | 1.98 | 2.04 | 3,143 | 778 |
| | Range | .045 | .0100 | 602 | 129 |

TABLE 9

| Sample | Specimen | Width | Thickness | Max Force | Strength |
|---|---|---|---|---|---|
| Number | ID | (in) | (in) | (lbs) | (psi) |
| 3916 | RF treated | 1.985 | 2.05 | 6,059 | 1,489 |
| 3917 | RF treated | 1.990 | 2.05 | 6,153 | 1,508 |
| 3918 | RF treated | 1.960 | 2.05 | 5,653 | 1,407 |
| | Mean | 2.055 | 2.05 | 5,955 | 1,468 |
| | Median | 2.05 | 2.05 | 6,059 | |
| | Std Dev | 0.016 | 0.000 | 266 | 54 |
| | Maximum | 1.99 | 2.05 | 6,153 | 1,508 |
| | Minimum | 1.96 | 2.05 | 5,653 | 1,407 |
| | Range | .03 | .000 | 500 | 101 |

As can now be appreciated, other applications of the method of this invention include, without limitation, scale removal and/or inhibition, cement strengthening, de-emulsification and hydrate removal and/or inhibition. For the latter two applications, the substance to be treated is a water-oil emulsion or one or more hydrates, respectively. With respect to de-emulsification, the radio signal treatment in accordance with this invention for de-emulsification operates to stretch the water droplets back and forth with the charge changes, the water droplets in oil-water emulsions presenting an unbalanced charged surface. The radio signal from an antenna or antenna array, when exposed to the oil containing the droplets of water, imparts an undulating motion to the droplet which destabilizes the surface of the water droplet and allows adjacent droplets to coalesce with it. The application of electromagnetic waves to such emulsions may be employed in storage tanks, emulsion treatment units and the like. In one aspect of this application, the emulsion treatment method comprises exposing an emulsion to one or more VHF frequencies preferably in the range of about 40 to about 50 MHz at a power level no greater than 1000 Watts for a period of time sufficient to cause oil-water separation. Various emulsions may be treated using this method. Non-limiting examples of such emulsions would include oil solutions of one or more phases of water in oil, and brine solutions in oil, or the like.

With respect to hydrate removal and/or inhibition, and without being bound to theory, it is believed the method of this invention directly effects hydrogen bonding. Hydrates are a function of hydrogen bonding, and treatment using the method of this invention should have and effect on the stability of the hydrate. Hydrates in hydrocarbon exploration operations present issues of undesirable ice formation under appropriate circumstances, which can block fluid flow in various settings in which fluid flow is critical to exploration and/or production. The electromagnetic wave treatment method of this invention can be applied to proactively prevent hydrate formation, or to treat existing hydrates. In one aspect of this application, the hydrate treatment method comprises exposing the treatment zone or an existing hydrate to one or more VHF frequencies in the range of about 40 to about 50 MHz range at power levels in accordance with the invention (1000 Watts or less). Various hydrates may be treated or inhibited using the method. Non-limiting examples of candidate hydrates include clathrate hydrates, e.g., methane hydrate and the like.

With respect to scale removal and/or inhibition, scale formation can occur in the well formation or in any production equipment exposed to mineral-containing formation fluids, especially near or at points of significant temperature or pressure change. The scale formation can reduce or block well fluid production and cause equipment to foul. In another aspect of the invention, a scale treatment method is provided, the method comprising exposing a treatment zone or existing scale to one or more VHF frequencies at power levels in accordance with the invention (1000 Watts or less). The VHF frequencies used may vary, depending at least in part upon the scale being treated, but for calcium carbonate and/or barium sulfate the frequency is preferably about 18 MHz. Various scale deposits may be treated or inhibited by this method. Suitable non-limiting examples of such scale are alkali earth and alkali earth metal salts (e.g., sodium chloride, calcium carbonate, barium sulfate, etc.), metal sulfides and/or other insoluble sulfides, and the like.

The invention enables cement strengthening in a wide variety of fields, including without limitation, the oil and gas exploration industry. Cement slurries according to this aspect of the invention are exposed to radio waves and/or microwave during setting/curing for a time and at a frequency sufficient to increase the crush strength of the set cement as compared to like cement set without the radio wave exposure. The frequencies used would fall in the range of about 1 MHz to about 100 MHz for radio waves and about 20 GHz to about 40 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

In another of its aspects, this invention enables improved chemical precipitation of target materials (e.g., iron) from completion fluids (e.g., high density brines). For example, a magnetic field (e.g., 3000 to 10000 gauss) may be applied to completion fluids while the fluids are exposed to radio wave and/or microwave frequencies in accordance with this invention, thereby causing the target material to flocculate and fall out of solution. The frequencies used would fall in the range of about 1 MHz to about 100 MHz for radio waves and about 20 GHz to about 50 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts. Suitable completion fluids to which this method could be applied would include any convention completion fluid taught in the literature, e.g., such as those taught in U.S. Pat. Nos. 4,967,838, 4,938,288, 4,780,220, 4,536,297, 4,521,316, 4,444,668 and 4,440,649, the disclosures of which are incorporated herein by reference.

The invention, in another of its aspects, also provides a method of inhibiting corrosion. Microwave wavelength exposure of a material or area in need of corrosion inhibition, within the broadest frequencies taught herein, are effective when provided substantially continuous exposure. The zone or material being treated should be underground or in a container. Without being bound to theory, it is believed that the sinusoidal, high frequency microwaves are believed to cause the corrosive material (e.g., metals such as simple steel and/or iron in production fluids) to oscillated between and oxidative and reductive state, by changing the spin state of electrons. The frequencies used would fall in the range of about 1 to about 100 MHz for radio waves and about 15 to about 50 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

The invention, in another of its aspects, also provides a method of reversing clay damage. Clay build-up in formation would be treatable using the processes of this invention. The frequencies used would fall in the range of about 1 to about 50 MHz for radio waves and about 15 to about 50 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

Still another aspect of the invention provides method of making or keeping iron sulfide soluble in acid used to treat well formations. It is believed, without being bound to theory, that this process works primarily because of the magnetic moment associated with the iron. Radio frequencies would be applied to the well formation during a conventional acid well treatment to soluabilize iron sulfide, and evolving hydrogen sulfide gas. In this process, a squeeze with the acid is conducted, forcing it into formation to break up the carbonates of scale plugging the well. The process is enhanced with the additional exposure of the formation to the electromagnetic waves as taught herein. The frequencies used would fall in the range of about 1 to about 50 MHz for radio waves and about 15 to about 50 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

Sediment in tank bottoms likewise may be treated using the exposure methods of this invention. However, preferably oxygen in the tanks to be treated would be purged, using an inert gas, to reduce explosion risks during treatment. This process breaks up sediment, which would be in solution at higher temperature, by increasing solubility of organics (e.g., paraffins) in solution. The frequencies used would fall in the range of about 20 to about 50 MHz for radio waves and about 20 to about 40 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

The invention also provides a method useful in cleaning injection wells. Injection wells would be treated in the same way as a production well, to facilitate removal of blockage inhibiting the performance of the injection well. The frequencies used would fall in the range of about 20 to about 80 MHz for radio waves and about 15 to about 30 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

In yet another aspect of this invention, the method can be used to enhance the performance of coil tubing tool systems intended to remove scale and other deposits from a well bore, well casing or tubing therein. The process comprises exposing the deposits to radio and/or microwaves in accordance with the processes described heretofore, while operating a coiled tubing agitating tool such as, e.g., a ROTOJET® tool available from BJ Services Company, Houston, Tex. See in this connection U.S. Pat. No. 6,607,607, the disclosure of which is incorporated herein by reference. In so doing, an increase in material that goes into solution for easy removal from the well bore is achieved. In one aspect of the invention, the tool itself would be modified to include the amplifier, tuner and antenna(s) in a plug connected to a power supply, so that the plug is capable of traveling down the tubing of the well as part of or in conjunction with the agitation tool. In this way, the system would expose the deposits to both physical agitation (e.g., stress cycling) and electromagnetic wave concurrently, to further enhance well bore cleanout. The frequencies used would fall in the range of about 10 to about 40 MHz for radio waves and about 20 to about 30 GHz for microwaves. Exposure duration could be 2 hours or more, or in the range of about 2 to about 24 hours. The power consumption during such method could be more or less than 1000 Watts, but in at least one aspect of the invention would be no more than about 1000 Watts.

It should now be appreciated that, for all of the foregoing applications of the present invention, for a given set of circumstances, the frequency or frequencies, duration of exposure and power level employed could vary and would normally be the subject of optimization within the skill of the ordinary artisan in this field having the benefit of this disclosure.

While the invention has been described here in the context of down hole applications in oil & gas well treatment, it will be appreciated by those of at least ordinary skill in the art, having the benefit of the present disclosure, that the invention has applications in many other areas in which the alteration of a one or more colligative or physical properties of a substance, under low power consumption conditions, could be desirable. Accordingly, the invention should not be construed as limited to the particular applications described in detail herein.

1.-20. (canceled)

21. A method comprising exposing a substance to a first type of electromagnetic waves generated by a first device, the frequency of the first type of electromagnetic waves being in the radio frequency range, the exposure taking place for a period of time and at a frequency sufficient to detectably alter at least one physical property of the substance as it existed prior to the exposure, wherein the substance is selected from the group consisting of a hydrate, a water and oil emulsion, clay, scale, cement, a completion fluid, tank sediment and iron sulfide.

22. A method according to claim 21, wherein the step of exposing the substance to the first type of electromagnetic waves is carried out at least while concurrently exposing the substance to a second type of electromagnetic waves generated by a second device, wherein the frequency of the second type of electromagnetic waves is in the microwave frequency range.

23. A method according to claim 22, further comprising
transmitting the electromagnetic waves at one or more radio frequencies through at least one first antenna
(i) connected to, or disposed within, a wellhead assembly, well casing or well tubing of a well; or
(ii) connected to, or disposed within, a pipeline or a tank,
the radio frequencies each being in the range of about 1 to about 900 MHz, wherein the process is conducted for a time sufficient to detectably alter at least one physical property of the substance within the well, pipeline or tank as the substance existed prior to the exposure.

24. A method according to claim 21, further comprising
transmitting the electromagnetic waves at one or more radio frequencies through at least one first antenna
(i) connected to, or disposed within, a wellhead assembly, well casing or well tubing of a well; or
(ii) connected to, or disposed within, a pipeline or a tank,
the radio frequencies each being in the range of about 1 to about 900 MHz, wherein the process is conducted for a time sufficient to detectably alter at least one physical property of the substance within the well, pipeline or tank as the substance existed prior to the exposure.

25. The method according to claim 24 further comprising
transmitting electromagnetic waves at a microwave frequency of at least about 24 GHz through at least one second antenna
(i) connected to, or disposed within, the wellhead assembly, well casing or well tubing of the well; or
(ii) connected to or disposed within a pipeline or a tank,
wherein the first antenna and the second antenna may be separate antennae or may be combined into the form of a single antenna.

26. The method according to claim 23 further comprising
transmitting electromagnetic waves at a microwave frequency of at least about 24 GHz through at least one second antenna
(i) connected to, or disposed within, the wellhead assembly, well casing or well tubing of the well; or
(ii) connected to or disposed within a pipeline or a tank,
wherein the first antenna and the second antenna may be separate antennae or may be combined into the form of a single antenna.

27. The method according to claim 25, wherein the microwave frequency is amplified to consume energy at a rate of no more than about 8 Watts.

28. The method according to claim 22, wherein the microwave frequency is amplified to consume energy at a rate of no more than about 8 Watts.

29. The method according to claim 28, wherein the first device consumes no more than about 1,000 Watts of power.

30. The method according to claim 21, wherein the first device consumes no more than about 1,000 Watts of power.

31. A method for treating and/or inhibiting hydrate formation, the method comprising carrying out the method in accordance with claim 21, wherein the substance comprises a hydrate, so that the amount of hydrate present in the substance is reduced.

32. The method of claim 31, wherein the frequency of the first type of waves is in the range of about 40 to about 50 MHz.

33. A method for de-emulsification of an emulsion, the method comprising carrying out the method in accordance with claim 21, wherein the substance comprises a water and oil emulsion, so that at least a portion of oil in the emulsion separates from water in the emulsion.

34. The method of claim 33, wherein the frequency of the first type of waves is in the range of about 40 to about 50 MHz.

35. A method for treating and/or inhibiting scale formation, the method comprising carrying out a method in accordance with claim 21, wherein the substance comprises scale, so that the amount of scale present or formed is reduced.

36. The method of claim 35, wherein the scale comprises calcium carbonate and/or barium sulfate, and the frequency of the first type of waves is about 18 MHz.

37. A method for increasing the crush strength of cement, the method comprising carry out the method in accordance with claim 21, wherein the substance comprises cement which has not set, so that upon setting the cement has an increased crush strength relative to its crush strength in the absence of the exposure.

38. A method for precipitating a target material from a completion fluid, wherein the method comprises carrying out the method in accordance with claim 21, wherein the substance comprises a completion fluid, while placing the fluid within a magnetic field, thereby causing the target material to flocculate and precipitate out of the fluid.

39. A method of inhibiting corrosion, wherein the method comprises carrying out the method in accordance with claim 21, wherein the substance is an substantially isolated target formation or system susceptible to corrosion, wherein the exposure is carried out substantially continuously, thereby reducing the corrosion which occurs in the target formation or system relative to corrosion in the absence of such exposure.

40. A method of reversing clay damage, wherein the method comprises carrying out the method in accordance with claim 21, wherein the substance comprises a clay.

41. A method of making or keeping iron sulfide soluble in an acid solution used to treat a well formation, the method comprising carrying out the method in accordance with claim 21, wherein the substance comprises iron sulfide in admixture with an acid solution in the formation, and the exposure is carried out during an acid treatment of the well formation so as to increase the amount of iron sulfide in solution with the acid.

42. A method of removing sediment from a tank bottom, the method comprising carrying out the method in accordance with claim 21, wherein the substance is the sediment, so as to increase the solubility of sediment component in a solution relative to the solubility of the same component in the absence of the exposure.

43. A method of treating an injection well, the method comprising carrying out the method according to claim 21, wherein the substance is blockage in the injection well, so that the fluid pressure is reduced during fluid injection into the well.

44. A method of enhancing the performance of a coiled tubing tool system for removing deposits from a well bore, the method comprising carrying out the method claim 21, while treating the well bore with the coiled tubing tool system, so as to increase the amount of deposits brought into solution for removal from the well bore.
